(19) United States
(12) Patent Application Publication
(10) Pub. No.: US 2003/0100022 A1
(43) Pub. Date: May 29, 2003

(54) Nucleic acid construct system and method utilizing same useful for identifying protein-protein interactions

(75) Inventors: Ami Aronheim, Binyamina (IL); Monika Hubsman, Beer Sheba (IL)

Correspondence Address: c/o ANTHONY CASTORINA, SUITE 207, 2001 JEFFERSON DAVIS HIGHWAY, ARLINGTON, VA, 22202, US

(73) Assignee: Technion Research and Development Foundation Ltd.

(21) Appl. No.: 09/777,856

(22) Filed: Feb. 7, 2001

Related U.S. Application Data
(60) Provisional application No. 60/220,153, filed on Jul. 24, 2000.

(51) Int. Cl. G01N 33/53; G01N 33/567; C12N 15/85

(52) U.S. Cl. 435/7.2; 435/455

(57) ABSTRACT

A method of identifying interactions between polypeptides is provided. The method is effected by: (a) expressing in a cell substantially lacking Ras activity: (i) a first polynucleotide, encoding a first polypeptide being capable of interacting with a plasmalemma of the cell; and (ii) a second polynucleotide encoding a second polypeptide fused to a cytoplasmic Ras mutant, the cytoplasmic Ras mutant being capable of the Ras activity if mobilized to the plasmalemma of the cell; and (b) detecting a presence or absence of the Ras activity in the cell, the Ras activity being indicative of a possible interaction between the first polypeptide and the second polypeptide.

This application claims priority from U.S. provisional patent application No. 60/220,153 filed Jul. 24, 2000

### FIELD AND BACKGROUND OF THE INVENTION

The present invention relates to a nucleic acid construct system and method utilizing same which can be used for identifying and/or characterizing protein-protein interactions.

The interaction between proteins or the protein subunits of a multimeric protein governs, or forms a part of, various cellular processes including signal transduction, enzymatic reactions such as DNA replication, intra and inter cellular transport and the like.

Uncovering a relationship between two or more proteins or between protein subunits can provide insight into the possible biological role of such proteins or protein subunits even in cases where no known functions are attributed thereto.

Protein-protein interactions can be transitory or stable in nature. For example, the formation of multimeric proteins, such as hemoglobin or the core RNA polymerase is dependent on stable interactions between two or more subunits. Although some of the subunits of such protein complexes are not tightly bound, activity is associated with a whole structure which is sometimes referred to as a ‘protein machine’ (Alberts and Miake-Lye 1992, Cell, 68:415-20).

Protein-protein interactions which are transient in nature typically govern processes such as enzymatic reactions. For example, the interactions between protein kinases, protein phosphatases, proteases and the like with their respective substrate proteins is transitory in nature. Such transient interactions are oftentimes more difficult to uncover in cases where the proteins or conditions responsible for transient interactions are not known.

Protein-protein interactions can have a number of different measurable effects. First, they can alter the kinetic properties of proteins. This is reflected by altered binding of substrate, altered catalysis, or altered allosteric properties of the complex (Monod 1966, Science, 154:475-83). Second, protein-protein interactions can result in the formation of a new binding site (Steitz et al. 1977, J Biol Chem, 252:4494-500). Third, the interaction can inactivate a protein; this is the case with the interaction of trypsin with trypsin inhibitor (Vincent and Lazdunski 1992, Biochemistry, 11:2967-77).

Protein-protein interactions can also change the specificity of a protein for its substrate. For example, the interaction of lacialbumin with lactose synthase lowers the Kₚ₁ for glucose a 1000-fold (Hill and Brew 1975, Adv Enzymol Relat Areas Mol Biol, 43:411-90).

An interaction between proteins can take place over a small or a large surface region depending on the proteins and type of interaction.

For example, interactions between Src homology 2 (SH2) domains and peptides containing a phosphotyrosine residue occurs at a specific binding pocket found in the SH2 domains (Waksman et al. 1993, Cell, 72:779-90).

In the case of the leucine zipper, interactions between long stretches of amino acids forming α-helices are observed (Ellenberger et al. 1992, Cell, 71:1223-37).

Since protein-protein interactions participate in various cellular processes understanding and characterizing protein-protein interactions is a key to understanding various cellular processes. As such, various physical, biochemical, molecular and genetic approaches for uncovering novel protein-protein interactions or better characterizing known interactions have been developed.

Biochemical/physical approaches for studying protein complexes are the most conservative, and have been in use since the 1970s.

One such technique is protein affinity chromatography which is based on the principle that a protein can be covalently coupled to a matrix such as Sepharose under controlled conditions and used to select proteins which specifically interact therewith under predetermined conditions. Despite it's sensitivity, this technique has several inherent limitations including the need for a pure protein preparation, and the lack of conformational preservation.

In affinity blotting, a technique analogous to affinity columns, proteins are separated via polyacrylamide gel electrophoresis (PAGE), transferred to a nitrocellulose membrane, and reacted with a candidate protein. This method is similar to immunoblotting, which uses an antibody as a probe. Although in this method complex mixtures of proteins, such as total-cell lysates, can be analyzed without any purification, conditions used for electrophoresis or blotting oftentimes lead to a loss of conformation which may be important for binding.

Cross-linking of protein complexes has also been used to uncover potential protein-protein interactions. Cross-linking can be effected via reagents such as a cleavable heterobifunctional photoactivatable cross-linking reagent (Denny and Blobel 1984, Proc Natl Acad Sci U S A, 81:5286-90). Such a reagent can be coupled to an isolated protein, which is then incubated in an appropriate extract and photoactivated to cross-link nearby proteins. Since the label is on the photo-activatable moiety of the cross-linking reagent, it is transferred to the cross-linked protein after cleavage of the cross-linking reagent. Although a significant number of cross-linking reagents are available, the major disadvantage of using this technique is that it detects the nearest ‘neighbors’ protein that may not be in direct contact.

The above discussed methods have been the basis of biochemical research for the last three decades. However such methods are unsuitable and as such inefficient for high throughput screening of protein-protein interactions.

As such, a variety of methods have been developed in order to enable screening of large polypeptide encoding libraries for proteins or peptides exhibiting protein-protein interactions. A distinct advantage of these methods over the biochemical methods described hereinabove is that the proteins or peptides identified can be directly related to the genes or polynucleotide fragments encoding such proteins or peptides.

A library of peptides or proteins (e.g., lambda library or a phage-display library) can be screened with a labeled protein or peptide of interest in order to uncover library clones exhibiting possible interactions therewith (Smith 1985, Science, 228:1315-7).

Of the various library screening methods, the yeast two-hybrid system is the most commonly used (Vidal and Endoh 1999, Trends Biotechnol. 17:374-81). The yeast two-hybrid system is an artificial transcription-based assay that relies on the principle that many proteins including transcriptional activators, include modular domains that can function independently. When individual domains are expressed separately and then brought into close proximity via non-covalent interactions, such domains can function collectively to reconstitute the activity of the intact protein. Transcriptional activators which include a DNA-binding domain (DBD) and a transcriptional-activation domain (AD) (Brent and Ptashne 1985) exhibit such properties. The DNA binding domain serves to target the activator to the specific genes that will be expressed, and the activation domain associates with other proteins of the transcriptional machinery to enable transcription to occur.

In the two hybrid system, two hybrids are constructed: the DNA-binding domain fused to a protein, X, commonly referred to as bait, and the transcription activation domain fused to a protein, Y, commonly referred to as prey. These two hybrids are expressed in a cell containing one or more reporter genes. If the X and Y proteins interact, they create a functional transcriptional activator by bridging the activation domain; this can be detected by expression of the reporter genes. While the assay has been generally performed in yeast cells, it works similarly in mammalian cells (Dalton and Treisman 1992, Cell, 68:597-612) and can be applied to any other eukaryotic cells. Two hybrid screens result in an immediate availability of the cloned gene for a binding protein identified. Because multiple clones that encode overlapping regions of proteins are often identified, the minimal domain for interaction may be elucidated from the initial screen (Iwabuchi et al. 1993, Oncogene, 8:1693-6). The two-hybrid system has several features that make it useful for analysis of protein-protein interactions. It is highly sensitive, detecting interactions of wide range of affinities, which are not detected in other methods (Van Aelst et al. 1993, Proc Natl Acad Sci U S A, 90:6213-7). Furthermore, interactions are detected within the native environment of the cell and hence no biochemical purification is required.

The progenitor two-hybrid system has evolved to facilitate in its use in a broader range of applications that address more complex protein interactions. Interactions dependent on post-translational modification, such as tyrosine phosphorylation which does not occur in yeast cells can be overlooked using the basic method. This problem was addressed by co-expressing the relevant tyrosine kinase with the bait and prey to facilitate tyrosine phosphorylation in yeast (Osborn et al. 1995, Biotechnology (N Y), 13:1474-8). An important consideration when utilizing this approach is whether the modifying enzyme is toxic when overexpressed in yeast, as a consequence of broad spectrum of substrates phosphorylated by the modifying kinase. This may be circumvented by using a modifier that exhibits a high degree of substrate specificity or by placing the modifier under the control of a regulated or weak promoter.

Additional modifications to the two-hybrid system have produced the three or tri-hybrid-system, which was developed in efforts to uncover interactions between several proteins. This modification resulted from the observation that many cellular proteins are multimeric. Hence, co-expression of a previously defined binding partner along with the bait might provide a stronger interaction interface that enables the identification of additional complex members (Polyak et al. 1994, Genes Dev, 8:9-22) (Tomashek et al. 1996, J Biol Chem, 271:10397-404).

The tri-hybrid approach has been used to identify enzymatic substrates recognized in the context of a ternary complex, such as those involved in regulating caspase activity against substrates (Van Criekinge et al. 1998, Anal Biochem, 263:62-6). In a related application, which has the potential to contribute to the advancement of therapeutic studies, a hormone or a drug can substitute for a protein in the role of ternary partner in a three-component interaction (Chiu et al. 1994, Proc Natl Acad Sci U S A, 91:12574-8). The primary weakness of this particular adaptation of the system is the permeability of yeast to ternary small molecules is questionable.

The two-hybrid system has also been used to evaluate interactions between bait proteins and minimal prey consisting of very small peptides fused to an activation domain. The utility of this approach is emphasized by its potential to define the minimal conserved sequences required for bait-prey interactions. In-line with this, a modified two-hybrid search, also termed reverse two-hybrid approach, can be used to indicate loss of protein-protein interaction. To this end the original method, was modified into a system in which activation of negatively counter-selectable markers can be evaluated (Vidal et al. 1996, Trends Biotechnol, 17:374-81). In addition to use in mutational haunts, this type of reverse selection can be used to screen drugs or peptide ligands that disrupt protein-protein interaction.

The recently developed Sos recruitment system (SRS) which also relies on the two hybrid approach, was designed to examine protein interactions that could not be detected in an ordinary two-hybrid search either because the protein of interest is transcriptionally active or associates with membranes and therefore does not localize to the nucleus (Aronheim et al. 1997 Mol. Cell Biol. 7, 3094-3102).

This strategy exploits basic properties of the Ras signal transduction pathway to provide means for positive selection. Yeast require Ras signaling to be viable, and in-turn Ras function is dependent on the activity of guanyl nucleotide exchange factors (GEFs), which activate Ras. The ability to manipulate the yeast genome, made it possible to generate temperature sensitive mutants for CDC25, the yeast GEF. These mutations can be complemented by the mammalian homologue Sos, when mobilized to the plasma membrane (Aronheim et al. 1994 Cell 78, 949-961). In the SRS strategy bait-prey interactions localizes Sos to the membrane, which triggers signaling and confers positive growth selection at a restrictive temperature.

Although the SRS system along with its derivatives, which are described hereinbelow, represent promising additions to the growing arsenal of strategies designed for investigating protein interactions, problems such as a high background of false positives which is inherent to such systems substantially hinders the use thereof.

Another alternative approach, which is an adaptive variant of the two hybrid system, relies on a reconstitution of an enzymatic reaction to assess protein interactions (Johnson et al. 1992, Embo J, 11:497-505). In this approach, protein X is fused to a carboxy-terminal fragment of ubiquitin, while protein Y is fused to a mutated amino-terminal fragment of ubiquitin. The interaction between the two molecules brings the amino and carboxy termini fragments of the protein into close proximity, thereby targeting the carboxy-terminal fusion protein for cleavage. The cleavage event, as monitored by western-blot analysis serves as the monitorable signal. To date, the system has not yet attained broad use and information regarding its efficiency and application is limited.

There is thus a widely recognized need for, and it would be highly advantageous to have, a method of identifying and/or characterizing protein-protein interactions devoid of the above limitations.

### SUMMARY OF THE INVENTION

According to one aspect of the present invention there is provided a method of identifying interactions between polypeptides comprising: (a) expressing in a cell substantially lacking Ras activity: (i) a first polynucleotide encoding a first polypeptide being capable of interacting with a plasmalemma of the cell; and (ii) a second polynucleotide encoding a second polypeptide fused to a cytoplasmic Ras mutant, the cytoplasmic Ras mutant being capable of the Ras activity if mobilized to the plasmalemma of the cell; and (b) detecting a presence or absence of the Ras activity in the cell, the Ras activity being indicative of a possible interaction between the first polypeptide and the second polypeptide.

According to further features in preferred embodiments of the invention described below, the method of claim 1, further comprising: (c) independently expressing in the cell substantially only the second polynucleotide to thereby determine if the Ras activity detected in step (b) is dependent on the interaction between the first polypeptide and the second polypeptide.

According to still further features in the described preferred embodiments an expression of the first polynucleotide and/or the second polynucleotide in the cell is separately regulatable such that step (c) is effected by downregulating an expression of the first polynucleotide and/or upregulating an expression of the second polynucleotide.

According to another aspect of the present invention there is provided a method of identifying interactions between polypeptides comprising: (a) expressing in cells substantially lacking Ras activity: (i) a first polynucleotide encoding a first polypeptide being capable of interacting with a plasmalemma of the cells; and (ii) a library of polynucleotides each encoding a distinct polypeptide fused to fused to a cytoplasmic Ras mutant, the cytoplasmic Ras mutant being capable of the Ras activity if mobilized to the plasmalemma of the cells; and (b) identifying a subset of cells exhibiting the Ras activity, the Ras activity being indicative of a possible interaction between the first polypeptide and a distinct polypeptide expressed in the subset of cells.

According to still further features in the described preferred embodiments the method further comprising isolating from each cell of the subset of cells a polynucleotide encoding the distinct polypeptide.

According to still further features in the described preferred embodiments an expression of the first polynucleotide and/or the library of polynucleotides in the cells is separately regulatable such that step (c) is effected by downregulating an expression of the first polynucleotide and/or upregulating an expression of the library of polynucleotides.

According to still further features in the described preferred embodiments the method further comprising: (c) independently expressing in the cells substantially only the library of polynucleotides to thereby determine if the Ras activity exhibited in the subset of cells is dependent on an interaction between the first polypeptide and the distinct polypeptide.

According to yet another aspect of the present invention there is provided a method of identifying interactions between polypeptides comprising: (a) expressing in cells substantially lacking Ras activity: (i) a library of polynucleotides each encoding a first polypeptide being capable of interacting with a plasmalemma of the cells fused to a second polypeptide; and (ii) a second polynucleotide encoding a cytoplasmic Ras mutant fused to a third polypeptide, the cytoplasmic Ras mutant being capable of the Ras activity if mobilized to the plasmalemma of the cells, and (b) identifying a subset of cells exhibiting the Ras activity, the Ras activity being indicative of a possible interaction between the third polypeptide and the second polypeptide expressed in each cell of the subset of cells.

According to still further features in the described preferred embodiments the method further comprising isolating from each cell of the subset of cells a polynucleotide encoding the second polypeptide.

According to still further features in the described preferred embodiments the method further comprising: (c) independently expressing in the cells substantially only the second polynucleotide to thereby determine if the Ras activity exhibited in the subset of cells is dependent on an interaction between the third polypeptide and the second polypeptide.

According to still further features in the described preferred embodiments an expression of the library of polynucleotides and/or the second polynucleotide in the cells is separately regulatable such that step (c) is effected by downregulating an expression of the library of polynucleotides and/or upregulating an expression of the second polynucleotide.

According to still another aspect of the present invention there is provided a method of identifying interactions between polypeptides comprising: (a) expressing in cells substantially lacking Ras activity: (i) a first library of polynucleotides each encoding a first polypeptide being capable of interacting with a plasmalemma of the cells fused to a second polypeptide; and (ii) a second library of polynucleotides each encoding a cytoplasmic Ras mutant fused to a third polypeptide, the cytoplasmic Ras mutant being capable of the Ras activity if mobilized to the plasmalemma of the cells; and (b) identifying a subset of cells exhibiting the Ras activity, the Ras activity being indicative of a possible interaction between the third polypeptide and the second polypeptide expressed in each cell of the subset of cells.

According to still further features in the described preferred embodiments the method further comprising isolating from each cell of the subset of cells polynucleotides encoding the second polypeptide and the third polynucleotides.

According to still further features in the described preferred embodiments the method further comprising: (c) independently expressing in the cells substantially only the second library of polynucleotides to thereby determine if the Ras activity exhibited in the subset of cells is dependent on an interaction between the third polypeptide and the second polypeptide.

According to still further features in the described preferred embodiments the first polypeptide is a native membrane protein.

According to still further features in the described preferred embodiments the cell substantially lacking Ras activity is a yeast cell or cells exhibiting a mutant Ras phenotype characterized by growth suppression under non-permissive conditions.

According to still further features in the described preferred embodiments the cytoplasmic Ras mutant is capable of complementing the mutant Ras phenotype if mobilized to the plasmalemma of the cell(s).

According to still further features in the described preferred embodiments the first polypeptide includes an amino acid sequence for plasmalemma targeting.

According to an additional aspect of the present invention there is provided a nucleic acid construct comprising a polynucleotide encoding a cytoplasmic Ras mutant under the transcriptional control of an inducible promoter.

According to yet an additional aspect of the present invention there is provided a nucleic acid construct library comprising a plurality of nucleic acid constructs each including a first polynucleotide region encoding a cytoplasmic Ras mutant translationally fused to a second polynucleotide region encoding a distinct polypeptide.

According to still further features in the described preferred embodiments each of the plurality of nucleic acid constructs further includes a promoter for directing the expression of the first and the second polynucleotide regions.

According to still an additional aspect of the present invention there is provided a nucleic acid construct system comprising: (a) a first nucleic acid construct including a first polynucleotide encoding a first polypeptide being capable of interacting with a plasmalemma of a cell in which it is expressed; and (b) a second nucleic acid construct including a second polynucleotide encoding a cytoplasmic Ras mutant fused to a second polypeptide.

According to still further features in the described preferred embodiments the first and the second nucleic acid construct each further include a promoter sequence for directing an expression of the first and the second polynucleotides in a eukaryotic cell.

According to still further features in the described preferred embodiments the first nucleic acid construct includes a first promoter sequence and the second nucleic acid construct includes a second promoter sequence, whereas the first and the second promoter sequences are independently selected from the group consisting of a constitutive promoter sequence and a inducible promoter sequence.

According to still further features in the described preferred embodiments the promoter is functional in eukaryotic cells.

According to still further features in the described preferred embodiments the promoter is an inducible promoter selected from the group consisting of Gal1 promoter, Yes2 promoter, Met425 promoter and a copper inducible promoter.

According to yet an additional aspect of the present invention there is provided a method of identifying polypeptides of interest comprising: (a) expressing in a cell substantially lacking Ras activity: (i) a first polynucleotide encoding a first polypeptide being capable of modifying a plasmalemma of the cell; and (ii) a second polynucleotide encoding a second polypeptide fused to a cytoplasmic Ras mutant, the cytoplasmic Ras mutant being capable of the Ras activity if mobilized to the plasmalemma of the cell; and (b) detecting a presence or absence of the Ras activity in the cell, the Ras activity being indicative of membrane mobilization of the second polypeptide resultant from a modification to the plasmalemma effected by the first polypeptide.

According to still further features in the described preferred embodiments modifying the plasmalemma of the cell is effected by modifying a protein or phospholipid component of said plasmalemma.

According to still further features in the described preferred embodiments the first polypeptide is PI3kinase.

The present invention successfully addresses the shortcomings of the presently known configurations by providing a high throughput method for accurately identifying protein-protein interactions between characterized or uncharacterizd proteins including interactions between native membrane proteins and protein or peptide ligands thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c illustrate the reverse RRS approach of the present invention. The membrane protein of interest is expressed in a Cdc25-2 yeast strain with no additional sequences. The Bait is than located at the membrane thus preserving its functional conformation. The bait protein is not expected to results in cell growth of Cdc25-2 yeast cells at the restrictive temperature (FIG. 1a). On the other hand, a protein expressed, for example, from a cDNA library, as a fusion with a cytoplasmic oncogenic Ras protein (FIG. 1b) is expected to mobilize to the plasma membrane upon interaction with the bait and as a result to enable cell growth at the restrictive temperature (FIG. 1c).

FIG. 2a illustrate a western blot analysis performed with anti-Myc antibodies. The regulation of the alcohol dehydrogenase (ADH, lanes 1-4), methionine (Met, lanes 5-7) and Gal1 (Gal, lanes 8-10) promoters was examined under three distinct medium conditions. The expression of myc-epitope Ras protein (myc-Ras, lanes 2-10) or Ras (lane 1) was detected with anti-myc antibodies. A migration distance of myc-Ras and protein size markers is indicated.

FIGS. 2b-c illustrate methionine dependent growth of transformants expressing protein interacting pairs. Cdc25-2 transformants expressing protein pairs under the control of regulatable promoter sequences were grown on glucose medium at 24° C. The plate colonies were replicated onto galactose containing plates with (FIG. 2c) or without (FIG. 2b) methionine and the plates were incubated at 36° C. Transformants expressing protein pairs under the control of methionine and Gal1 promoters, respectively, are able to grow only on galactose containing plate lacking methionine (FIG. 2b).

FIG. 3 is a flow chart illustrating the screening method of the present invention. Cdc25-2 cells are co-transfected with the membrane bait and a cDNA library fused to Ras. The expression of the bait and Ras-prey proteins is controlled by the methionine and Gal1 promoters, respectively. Transformants are selected on glucose minimal plates lacking leucine and uracyl at 24° C. Following 5-7 days, plates are replica plated onto galactose containing plates either containing or lacking methionine and incubated at 36° C. Transformants that exhibit efficient growth on the plates lacking methionine and no growth on the plate containing methionine are selected on a glucose plate and grown at 24° C. The selected transformants are retested for their methionine dependent growth. Those transformants that pass the secondary methionine dependent test are considered candidates and are further pursued. DNA is extracted from candidates and library plasmid is identified by restriction digest. The DNA is used to retransform Cdc25-2 cells with either the specific bait or non-specific bait.

FIGS. 4a-b illustrate a secondary methionine dependency test for the ChpAcΔN interacting candidates. Cdc25-2 cells were co-transfected with met expression plasmid encoding for myc-ChpAcΔN protein bait (Met-ChpAcΔN) and pYes expression plasmid encoding for oncogenic cytoplasmic Ras protein fused to rat pituitary cDNA library sequences. Two hundred thousand transformants were plated on 20 glucose plates lacking leucine and uracyl and incubated at 24° C. Following the first replica plating about 5% of the transformants exhibited efficient growth on galactose containing medium at the restrictive temperature. Only thirty clones showed preferential growth on the galactose containing plate lacking methionine. Subsequently, those transformants (only twenty are shown) were subjected to a secondary methionine test which resulted the identification of two clones (indicated by *) that did not grow on the galactose plate containing methionine (FIG. 4b), but grew efficiently on galactose devoid of methionine (FIG. 4a).

FIGS. 5a-b illustrate a specificity test for the library plasmids extracted from candidates clones 1 and 2. DNA plasmids isolated from these candidate clones were digested via restriction enzymes and the library plasmid was identified. The plasmid was used to co-transfect Cdc23-2 cells with: methionine expression vector encoding for the original bait (ChpAc), N terminal deleted Chp (ChpAcΔN), methionine expression vector (Met) and methionine expression vector encoding for full length myristoylated Chp activated (Met-M-ChpAc). Transformants grown on glucose plates at 24° C. (FIG. 5a) were replica plated onto plates containing galactose and lacking methionine and incubated at 36° C. (FIG. 5b).

### DESCRIPTION OF THE PREFERRED EMBODIMENTS

The present invention is of a nucleic acid construct system and method which can be used to identify and/or characterized protein-protein interactions.

Specifically, the present invention can be used to identify protein or peptide ligands of membrane proteins which are provided in a native configuration.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As used herein, the phrase “protein-protein interaction” refers to all interaction between proteins, polypeptides or short peptides or an interaction between combination thereof, which interaction lends to the formation of a transient or stable complex.

Herein the phrase “plasma membrane” and the term “plasmalemma” are used interchangeably to refer to the lipid bi-layer which surrounds and defines the cytoplasm.

As used herein the phrase “cytoplasmic Ras mutant” refers to a Ras mutant protein lacking a membrane targeting signal and thus incapable of self targeting to the plasma membrane. The oncogenic cytoplasmic Ras protein described in the Examples section is an example of a cytoplasmic Ras mutant.

The two hybrid system is widely used for identifying protein-protein interactions. However, screening via the two hybrid system is oftentimes limited to proteins which can be efficiently transported to the nucleus since nuclear localization is a prerequisite for binding qualification.

Although the two hybrid system has been used to identify proteins interacting with the cytoplasmic tail of different membrane proteins having a single transmembrane domain, or with membrane associated proteins such as CTPase, such a system is incapable or inefficient in identifying proteins ligands of membrane proteins especially in cases where the membrane protein includes several transmembrane domains.

Several methods were developed for the identification of protein-protein interaction outside the nucleus [25, 26], however, their applicability for integral membrane proteins and the screening feasibility are yet to be proven.

A recent study describes a novel approach for monitoring integral membrane protein-protein interactions in yeast [27]. This approach is based on disruption of a G-protein signaling cascade monitored by inhibition of a β-galactosidase reporter based assay. Although an interaction between known proteins was demonstrated by this approach, an efficient screening protocol is yet to be developed. One obvious problem that may arise is the ability to monitor decrease in reporter activity and selection of positive clones while screening library constituents.

An alternative approach for studying protein-protein interactions in yeast cytoplasm was recently developed [8, 15]. This approach which is termed Ras recruitment system (RRS) is based on the translocation of a cytoplasmic Ras to the plasma membrane mediated by protein-protein interaction. Such Ras membrane recruitment results in activation of a viability pathway in yeast and a detectable phenotype. The RRS approach suffers from a severe limitation in that fusion of a membrane protein to Ras will result in its membrane translocation independent of protein-protein interaction and a high false positive signal.

While reducing the present invention to practice, the present inventors have substantially improved on the RRS approach to thereby eliminate the high false positive signal characterizing that system.

Thus according to one aspect of the present invention there is provided a method of identifying interactions between polypeptides.

The method includes a first step in which a first polynucleotide encoding a first polypeptide capable of interacting with a plasmalemma of the cell and a second polynucleotide encoding a second polypeptide fused to a cytoplasmic Ras mutant are expressed in a cell substantially lacking Ras activity. The cytoplasmic Ras mutant is characterized by an ability to provide Ras activity in the cell if mobilized to the plasmalemma thereof.

The method further includes the step of detecting a presence or absence of Ras activity in the cell, such Ras activity is indicative of a possible interaction between the first polypeptide and the second polypeptide.

The cell lacking Ras activity described herein can be any cell including mammalian and insect cells which can be Ras mutated provided restoration of Ras activity produces a detectable signal or change in a phenotype.

Preferably, the cell is a Cdc25-2 yeast cell which is incapable of growth under restrictive conditions characterized by a temperature in the range of 32-40° C. In such cells translocation of the cytoplasmic Ras mutant to the plasmalemma enables growth under such restrictive conditions.

The first and second polynucleotide sequences are expressed in the cell from an nucleic acid construct system which includes a first expression construct for expressing the first polynucleotide sequence, and a second expression construct for expressing the second polynucleotide sequence.

As such, each of the first and second constructs also includes a promoter sequence which serves for directing the transcription of the first and second polynucleotides.

To ensure efficient selection of double transformants, each construct of the nucleic acid construct system also includes a distinct selection marker. For example, constructs used for yeast transformation, can include auxotrophic markers such as those described in the Examples section which follows.

To generate the nucleic acid constructs of the present invention, the polynucleotide sequences encoding the first polypeptide and the second polypeptide-Ras fusion can be ligated into a commercially available expression vector system suitable for transforming eukaryotic cells and for directing the expression of these factors within the transformed cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

For mammalian expression, suitable expression vectors include, but are not limited to, pcDNA3, pcDNA3.1(+/−), pZeoSV2(+/−), pSecTag2, pDisplay, pEF/myc/cyto, pCMV/myc/cyto, pCR3.1, which are available from Invitrogen, pCI which is available from Promega, pBK-RSV and pBK-CMV which are available from Stratagene, pTRES which is available from Clontech, and their derivatives.

The nucleic acid constructs of the present invention can be introduced into a mammalian cells via any standard mammalian transformation method. Such methods include, but are not limited to, direct DNA uptake techniques, and virus or liposome mediated transformation (for further detail see, for example, “Methods in Enzymology” Vol. 1-317, Academic Press).

For yeast expression, suitable expression vectors include, but are not limited to, pPIC, pGAP or pYES which are available from Invitrogen, pESC which is available from Stratagene or pYEX which is available from Clontech, and their derivatives.

The nucleic acid constructs of the present invention can be introduced into yeast cells via any standard yeast transformation method. Such methods include but are not limited to electroporation, calcium chloride transformation and rubidium chloride transformation.

The first polypeptide can be a native membrane protein which when expressed in the cell naturally associates with the plasma membrane either through inner layer binding/anchoring or via bi-layer traversion one or more times. Examples of native membrane protein include components of cell receptors, protein components of ion channels, transporters, and the like.

It will be appreciated in this case, that native membrane protein using native proteins enables to uncover protein ligands for

Alternatively, the first polypeptide can be a soluble peptide or protein fused to membrane targeting sequence such as a myristoylation signal sequence.

The second polypeptide can be a previously characterized protein or a fragment thereof, an uncharacterized protein or a fragment thereof or a combinatorial peptide.

It will be appreciated that expression of a second polynucleotide which encodes cytoplasmic Ras mutant fused to a membrane protein can result in membrane mobilization of Ras which is independent of interaction with the expressed first polypeptide thus resulting in generation of a false positive signal.

Thus, the method of the present invention further includes the step of independently expressing in the cell substantially only the second polynucleotide to thereby determine if the Ras activity detected results from specific interaction between the first polypeptide and the second polypeptide or simply from the mobilization of Ras to the plasmalemma independent of such interaction.

Independent or selective expression of the second polypeptide-Ras fusion can be achieved via any one of several approaches.

Control cells can be transformed with an expression construct which includes the second polynucleotide thus expressing only the second polypeptide-Ras fusion. Ras activity in such cells can be compared with cells which express both the first and second polynucleotides to thereby determine dependency of the Ras activity on protein-protein interactions.

Alternatively and preferably, the expression of the first and optionally the second polynucleotides can be regulated in the cell by for example, using inducible/repressible promoters. Examples of inducible/repressible promoters suitable for use in mammalian cells include, but are not limited to, the MMTV promoter. Tet-on and Tet-off (available from Clontech) and POP13 (available from Stratagene). Examples of inducible/repressible promoters suitable for use in yeast cells include, but are not limited to, the Gal1 promoter, the Yes2 promoter (available from Invitrogen), the Met425 promoter described by Mumberg et al. 1994 and the copper inducible promoter described by Butt et al. 1984 Proc. Natl. Acad. Science USA 81, 3332-3336.

For further detail on the use of inducible/repressible promoter sequences in selective expression schemes, refer to Example 1 of the Example section which follows.

Thus, the present invention provides a method and an expression construct system which can be used to identify and/or study protein-protein interactions.

The present invention can also be used to screen a library of proteins or peptides which are fused to the cytoplasmic Ras mutants for proteins capable of interacting with a membrane associated target.

Thus, according to another aspect of the present invention there is provided a method of identifying interactions between polypeptides.

The method is effected by expressing in cells substantially lacking Ras activity: a first polynucleotide encoding a first polypeptide capable of interacting with a plasmalemma of the cells along with a library of polynucleotides each encoding a distinct polypeptide fused to a cytoplasmic Ras mutant.

Following expression which can be effected as described hereinabove, the method further includes the step of identifying a subset of cells exhibiting Ras activity and isolating from each cell of the subset of cells a polynucleotide encoding the distinct polypeptide to thereby isolate a putative peptide or protein ligand of the first polypeptide.

It will be appreciated that a reverse library in which polynucleotides encoding a first polypeptide capable of interacting with a plasmalemma of the cells fused to a distinct polypeptide can be screened against a Ras-fused polypeptide is also envisaged by the present invention.

The library of polynucleotides utilized by the present invention can encode previously characterized proteins or fragments thereof, uncharacterized proteins or fragments thereof or combinatorial peptides.

The present invention can also be used to screen constituents of two distinct or similar libraries by constructing a first library fused to a polypeptide capable of interacting with a plasmalemma of the cells and a second library fused to the cytoplasmic Ras mutant.

Thus, the present invention provides a novel cell based approach for uncovering and/or studying protein-protein interaction. The present invention can be used to identify interactions between previously characterized proteins, to identify interactions between uncharacterized proteins, protein fragments, combinatorial peptides and the like, to determine the effect of mutations or substitutions on previously characterized protein-protein interactions and to screen libraries of proteins or peptides for putative protein pairs or complexes in a high throughput manner.

In addition, and in contrast to the prior arts the present invention enables screening of protein or peptide libraries for interactions with native membrane proteins while such proteins are provided in their native configuration. This unique feature of the present invention enables to uncover the peptide or protein ligands of ion channel and transporter proteins or cell receptor proteins which are involved in cellular processes governing cellular transition into a cancerous state. Once identified, such peptide or protein ligands can be further studied so as to determine their potency as possible anti-cancer drug leads.

In another similar approach, the present invention can also be used to screen polypeptides which are recruited to the plasmalemma of a cell following modification of a plasmalemma constituents, effected by, for example, an enzyme such as a kinase, phosphatase or the like.

Thus according to another aspect of the present invention there is provided a method of identifying polypeptides of interest. The method is effected by first expressing in a cell substantially lacking Ras activity a first polynucleotide encoding a first polypeptide capable of modifying a plasmalemma of the cell and a second polynucleotide encoding a second polypeptide fused to a cytoplasmic Ras mutant.

Following expression, the presence or absence of the Ras activity in the cell is detected.

Such Ras activity is indicative of membrane mobilization of the second polypeptide, which mobilization results from a modification to the plasmalemma effected by the first polypeptide.

For example, by expressing PI3kinase in cells along with a polypeptide, or a library of polypeptides fused to the cytoplasmic Ras mutant, one can uncover polypeptides which interact with PI3kinase-modified plasmalemma constituents.

PI3kinase is an enzyme which phosphorylates phospholipids and as such changes the phospholipid content of the cellular membrane (Isakoff et al EMBO J. 17 5374-5387). Such a change results in recruitment of proteins to the membrane via interactions mediated by PH domains such as PDKI and PKB. Thus, by using activated PI3Kinase one can isolate peptides or proteins which are capable of such interactions.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley mid Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1999); methodologies as set forth in U.S. Pat. Nos. 4,666,828; 4,683,202; 4,801,531; 5,192,659 and 5,272,057; “Cell Biology: A Laboratory Handbook”. Volumes I-III Cellis, J. E., ed. (1994); “Current Protocols in Immunology” Volumes I-III Coligan J. E., ed. (1994); Stites et al. (eds) “Basic and Clinical Immunology” (8th Edition), Appleton & Lange, Norwalk, Conn. (1994) Mishell and Shiigi (eds), “Selected Methods in Cellular Immunology”, W. H Freeman and C., New York (1980); available immunoassays are extensively described in the patent and scientific literature, see, for example, U.S. Pat. Nos. 3,791,932; 3,839,153; 3,850,752; 3,880,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986), “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

The present invention provides a system and method for identifying protein-protein interactions. The present invention, designated herein as “reverse Ras recruitment” is based on the fact that Ras localization to the plasma membrane (plasmalemma) is crucial for its function [8, 13]. Similar to the RRS system, expression of cytoplasmic Ras in yeast does not complement mutation in the Ras guanyl nucleotide exchange factor, CDC25-2 [14]. However, membrane bound mammalian Ras can efficiently complement this CDC25-2 mutation [9, 15]. Ras membrane translocation which results in the present invention from protein-protein interaction [8] can be readily monitored in a Cdc25-2 yeast strain since it facilitates cell growth at restrictive temperatures [8].

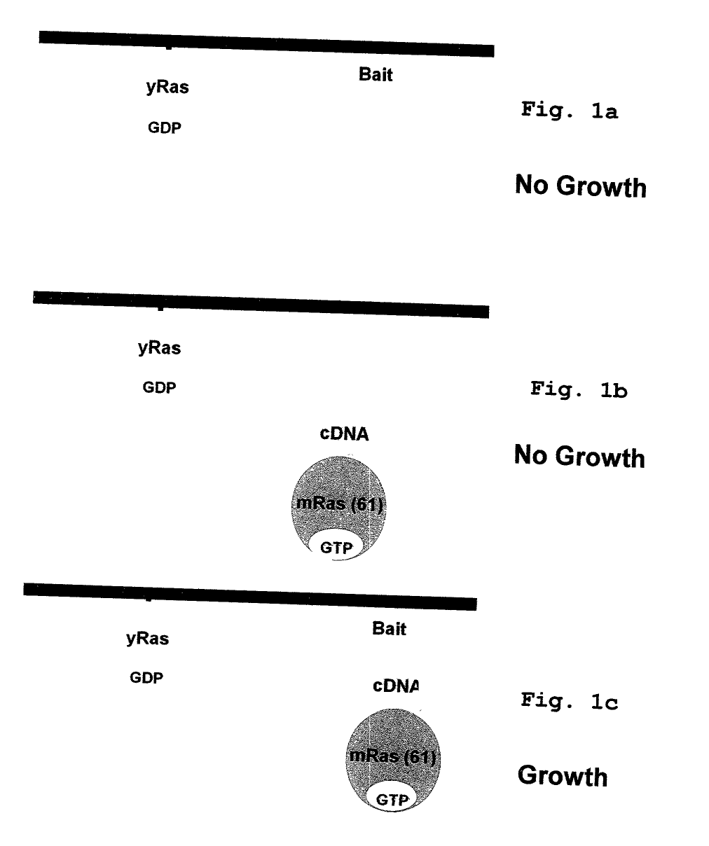

A schematic diagram describing the basis of the present invention is depicted in FIGS. 1a-c. In principle, the expression of a cDNA encoding for a membrane protein is expected to locate the protein at its natural environment i.e. the plasma membrane (FIG. 1a). ln order to identify proteins capable of interacting with the membrane protein (the “bait”), a cDNA encoding a protein of interest or a cDNA library cam be fused to the cytoplasmic Ras mutant coding sequence to express a fusion protein which acts as “prey” (FIG. 1b). The expression of either the bait or the prey alone is not expected to complement Cdc25-2 mutation. However, upon protein-protein interaction between the membrane protein and the fusion protein (Ras-cDNA), Ras is mobilized to the plasma membrane thus resulting in efficient growth of Cdc25-2 cells under restrictive temperatures (FIG. 1c).

### MATERIALS AND METHODS

Plasmids:

Rat pituitary cDNA library expression plasmid (prey): Rat pituitary cDNA which was excised from a rat pituitary library [9] via EcoRI-XhoI digestion was re-ligated into a pYes2 expression vector (Invitrogen Inc.) downstream of an oncogenic cytoplasmic Ras protein coding sequence (SEQ ID NO:1) which lacks a membrane localization signal and stop codon (cytoplasmic Ras mutant). The library generated represented over 4×10⁶ independent cloning events.

Methionine expression plasmic (bait): The p425-Met25 expression vector utilized herein was previously described in [10]. The Chp cassette (described below) was subcloned into the HindIII-XhoI cloning sites of p425-Met25.

A ChpAcΔN cDNA fragment (nucleotides 97-711 of SEQ ID NO:2) was PCR amplified from a ChpAc cDNA template (SEQ ID NO:2) using forward primer: 5′-CGGAATTCAAATGCGTGCTGGTGG-3′ (SEQ ID NO:3) and reverse primer: 5′-CCAAGCTATTTAGGTGACAC-3′ (SEQ ID NO:4). The resulting PCR product included a 5′ EcoRI site and a 3′ Xho site.

This PCR product and a full length Chp PCR product were each fused to a myc epitope tag (SEQ ID NO:5) and subsequently digested and cloned into a pMet (p425-Met25) expression plasmid to generate pMet-M-ChpAcΔN which encodes an N-terminal truncated Chp fused to a v-Src myristoylation signal or pMet-M-ChpAc which encodes a full length Chp fused to a v-Src myristoylation signal (SEQ ID NO:6). In addition, the ChpAc PCR product was also cloned into a YesM?PolyA plasmid described in [11].

The Pak65 plasmid used herein was constructed as previously described [11].

Transformant selection: To be able to select for double transformants, the prey and bait expression plasmids were designed so as to be able to complement the uracyl and leucine auxotrophy, respectively.

Yeast manipulations: All yeast transfection and manipulations were carried out by standard procedures as described in [8, 9]. Plasmid DNA was recovered from candidates as described [12]. Methionine was added to the growth medium to a concentration of 0.66 mM in order to efficiently repress the methionine promoter [10].

Cell extract and Western blot analysis: A 3 ml culture was grown over night at 24° C. Cells were recovered by centrifugation and resuspended in 0.5 ml of H₂O. Alkali lysis was performed at 4° C. by addition of 1.85 M NaOH/7.4 % b-mercaptoethanol for 10 minutes. Protein was recovered by the addition of 40 ml of 100% trichloroacetic acid to the lysed cells followed by 10 minutes incubation at 4° C. and 10 minutes centrifugation. The precipitated extract was washed once with ice-cold acetone and air dried. The pellet was solubilized in an sodium dodecyl sulfate (SDS) protein sample buffer, electrophoresed on 12.5% SDS-PAGE gel and immobilized onto nitrocellulose. The filter was incubated in 10% low-fat milk in PBS overnight followed by incubation with anti-Myc monoclonal antibodies (9E10; Babco Inc.) for 12 hours. Following incubation, the filter was washed twice in PBS and incubated with goat anti-mouse antibody conjugated to horse reddish peroxidase (Transduction laboratories Inc.). Following extensive washing in PBS, a chemiluminescent reaction (Super signal; Pierce Inc.) was performed and the filter was exposed to autoradiography.

### EXAMPLE 1

### The Reverse Ras Recruitment System Approach

Development of double inducible promoters system: In order to be able to screen for novel proteins which interact with the membrane bait, a cDNA expression library fused to cytoplastic Ras mutant was constructed (FIG. 1B). The library inserts were placed under the control of the Gal1 promoter.

When grown in a galactose containing medium, 5% of the yeast transformants exhibited efficient cell growth at the restrictive temperature independent of the expression of a specific protein bait.

This relatively high background, required the development of an approach enabling efficient selection for yeast transformants that exhibit growth which is solely dependent on protein-protein interactions.

Thus, while reducing the present invention to practice, a novel expression plasmid was generated This plasmid places the membrane bait protein under the control of a distinct inducible promoter, such as Met [10] which is induced when cells are grown in a medium lacking methionine.

It will be appreciated that to be able to identify cells in which the bait-prey interaction leads to cell growth under a restrictive temperatures, it is essential to be able to tightly control the expression of the bait plasmid independent of the expression of the prey expression plasmid.

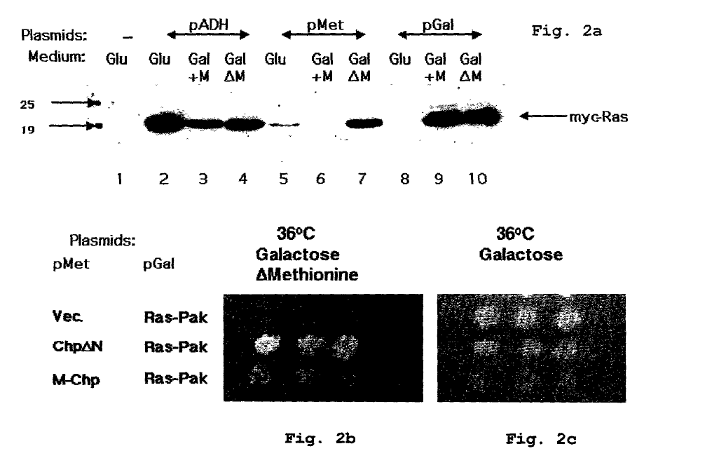

Thus, prior to screening, the expression from the Gal1 and methionine promoters was quantitated using dedicated expression constructs. Plasmids encoding for a myc-epitope tag-Ras protein placed under the control of alcohol dehydrogenase (ADH), Gal1 and Met promoters were used for determining expression levels under induced and non-induced (repressed) conditions. The plasmids were used to transform Cdc25-2 yeast cells and the transformants were selected and grown in three different liquid medium containing either glucose or galactose medium in the presence and absence of methionine. Extract derived from these transformants was subjected to SDS-PAGE and Western blot analysis using anti-Myc antibodies (FIG. 2a). This analysis revealed that the expression levels of myc-Ras protein under the Gal1 and the Met promoters are relatively similar when the cells are grown in galactose medium in the absence of methionine. However, the addition of methionine to the medium completely abolished Ras expression from the Met promoter. The Gal1 promoter is completely repressed when the cells were grown on glucose medium. This analysis strongly suggests that the expression of proteins under the control of the Gal1 and methionine promoters could be tightly regulated. Once induced, the expression levels are comparable to the expression obtained from the constitutive alcohol dehydrogenase promoter (ADH).

### EXAMPLE 2

### Positive Control

An interaction between known interacting protein pairs placed under the control of Gal1 and Met promoters was tested in order to assess the ability of the present invention to accurately identify protein-protein interactions.

Polynucleotide sequences encoding a novel small GTPase, designated Chp [11] fused to v-Src myristoylation signal (M-Chp) and its protein ligand Pak65 fused to Ras (Ras-Pak) were placed under the control of Met and Gal1 promoters respectively. Transformants expressing both proteins exhibited efficient growth only when grown on galactose containing medium in the absence of methionine (FIG. 2b). Similar results were obtained when Ras-Pak and M-Chp were expressed under the control of Met and Gal1 promoters respectively. The ability to detect interaction between Ras-Pak and Chp, using the present invention, is absolutely dependent on membrane localization of Chp through the v-Src myristoylation signal. This is due to the fact that Chp, unlike all Rho-GTPases, does not contain the typical C-terminal consensus CAAX box (SEQ ID NO:7) [16]. Surprisingly, it was uncovered that transformants expressing the Chp protein lacking the N-terminal domain (ChpAcΔN) along with the Ras-Pak protein, exhibited efficient growth at the restrictive temperature (FIG. 2b) This interaction, no longer required the v-Src myristoylation signals. Suggesting, that upon truncation of the N-terminal of Chp, the protein translocates to the plasma membrane.

### EXAMPLE 3

### Screening for Proteins Interacting With ChpAcΔN

The fact that Chp was able to rescue Cdc25-2 cells when expressed with Ras-Pak and the observation that both the Met and Gal1 promoters can be tightly regulated confirmed that the present invention can be utilized for identifying protein-protein interactions.

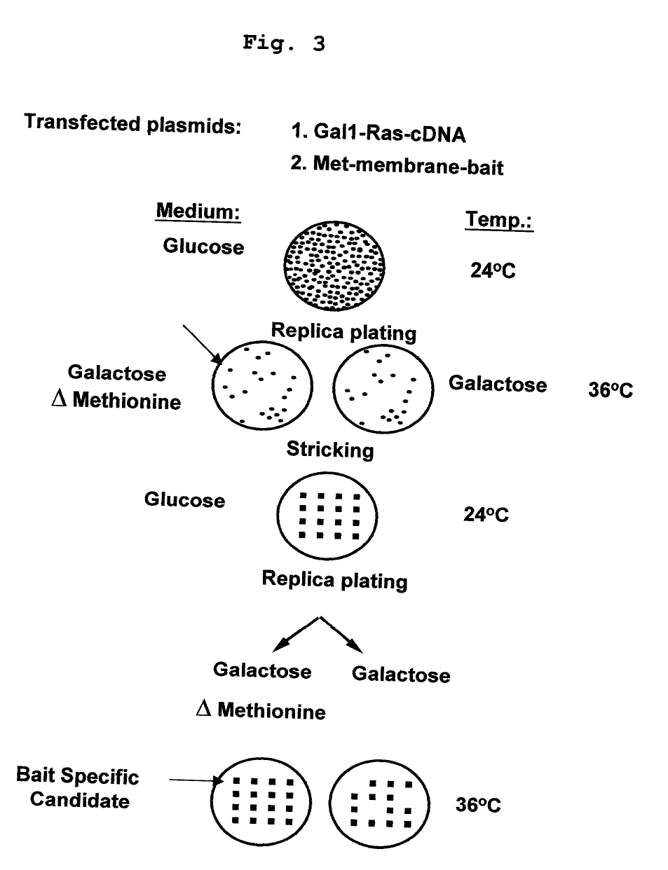

A flow chart depicting a library screening procedure according to the present invention is depicted in FIG. 3. Approximately 200,000 transformants (5% of the complexity of a rat pituitary library) were plated on glucose plates containing the appropriate growth selection compounds and subsequently replica plated on to galactose containing plates either in the absence or presence of methionine. Thirty yeast colonies that exhibited efficient growth on the galactose plate lacking methionine as compared to the growth obtained on the plate containing methionine were selected and exposed to a secondary growth dependent test on galactose containing plates in the presence or absence of methionine.

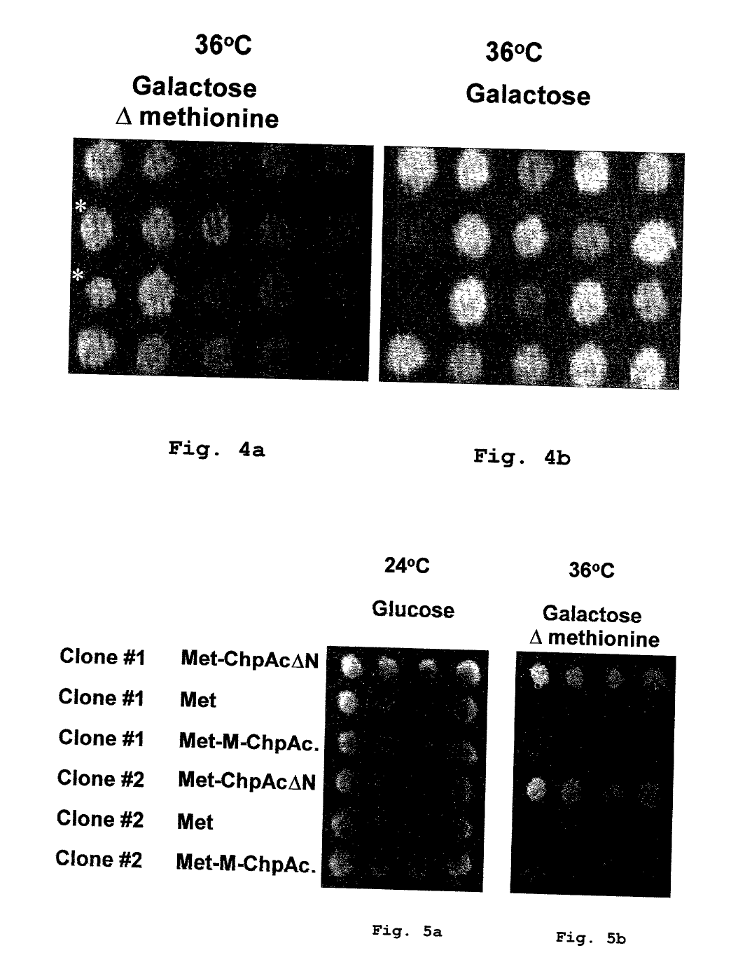

Two out of the 30 colonies selected exhibited reproducible growth on galactose medium in the absence of methionine but not on galactose medium containing methionine (FIGS. 4a-b). Plasmid DNA extracted from these clones was used to transform yeast cells along with the specific bait (ChpAcΔN) a Met expression plasmid lacking an insert (Met empty) or a myristoylated full length activated Chp (M-ChpAc.) (FIGS. 5a-b).

Both clone 1 and 2 exhibited efficient growth under the restrictive temperature in the presence of the original ChpAcΔN bait (FIG. 5b) while exhibiting little or no growth in the presence of the Met empty expression plasmid (FIG. 5b).

In addition, transformants expressing clone 1 and the myristoylated full length activated Chp did not exhibit growth under restrictive conditions while transformants expressing clone 2 and this myristoylated full length activated Chp exhibited growth although to a lesser degree than the ChpAcΔN transformants (FIG. 5b).

Collectively, these results indicate that the growth of the transformants isolated indeed was dependent on the expression and co-interaction of both the bait and the prey encoding proteins.

Thus the present invention provides a novel approach for identifying and/or studying protein-protein interactions. Such an approach can be utilized for uncovering protein ligands of membrane receptors, ion channels, and transporters that span the plasma membrane.

In contrast to prior art methods, the present invention enables to identify protein ligands of membrane proteins while such membrane proteins are provided in their natural environment which preserves their unique three dimensional structure and binding surfaces arrangement.

As shown herein, the present invention efficiently identifies novel protein-protein interactions. Using ChpAcΔN as bait, two distinct cDNAs encoding for proteins that exhibit specific interaction therewith were identified.

Clone 1 encodes a placenta growth factor (PIGF accession number L40030). Although an interaction between a growth factor and Chp is not readily explainable at present, the binding sites participating in such an interaction can contribute to characterization of binding surfaces and to further elucidation of the role of each of the proteins.

Clone 2 encodes a yet uncharacterized protein (mouse EST accession number AA789582). Although the biological importance of this interactions is yet to be determined, such an interaction provides insight as to the possible function of this uncharacterized protein.

The present invention provides an efficient method of exploring interactions between novel proteins and membrane multiple targets. For example, the hetero-oligomerization between different members of G protein-coupled receptors was recently demonstrated for the neurotransmitters somatostatin and dopamine receptors [28]. This interaction results in their enhanced functional activity. It will be interesting to explore the protein interaction with such oligomers in the presence of their corresponding ligands.

The use of a yeast cell system for exploring mammalian protein-protein interactions provides numerous benefits.

Although the existence of yeast homologues for mammalian membrane proteins can result in interactions between the prey and a yeast homologues thus providing positive results independent of an interaction with the bait, manipulation of the yeast genome by gene knockout can overcome this problem.

Moreover, the existence of yeast pathway mutants can be used for complementation assays, with would test the functionality of the protein prior to screening. This may provide evidence that the membrane protein indeed fully preserves its activity and potentially its protein binding surfaces. In addition, G-protein coupled receptor, for example, can be used to screen for protein binding either in the presence or absence of their specific ligands or agonists.

Moreover, in many instances where a prey is identified by the present invention, it is often useful to screen for additional proteins interacting with the prey protein. For example, using the present invention one can simply use the prey protein as a bait and perform screening with a Ras fused library of the present invention.

Although the invention has been described in conjunction with specific embodiments thereof, it is evident that many alternatives, modifications and variations will be apparent to those skilled in the art. Accordingly, it is intended to embrace in such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

### REFERENCES

(Additional References Cited Within Text)

1. Mendelsohn, A. R. and Brent, R. Protein interaction methods-toward an endgame. (1999) Science, 284: 1948-1950.

2. Blackstock, W. P. and Weir M. P. Proteomics' quantitative and physical mapping of cellular proteins. (1999) Trends Biotechnol., 17: 121-127.

3. Dunham, I., Shimizu, N., Roe, B. A., Chissoe, S., Hunt, A. R., Collins, J. E., Bruskiewich, R., Beare, D. M., Clamp, M., Smink, L. J., et al. The DNA sequence of human chromosome 22. (1999) Nature, 402: 489-95.

4. Goffeau, A., Barrell, B. G., Bussey, H., Davis, R. W., Dujon, B., Feldmann, H., Galibert, F., Hoheisel, J. D., Jacq, C., Johnston, M., et al. Life with 6000 genes. (1996) Science, 274:546,563-567.

5. Uetz, P., Giot, L., Cagney, G., Mansfield, T. A., Judson, R. S., Knight, J. R., Lockshon, D., Narayan, V., Srinivasan, M., Pochart, et al. A comprehensive analysis of protein-protein interactions in Saccharomyces cerevisiae. (2000) Nature, 403: 623-627.

6. Fields, S. and Song, O. K. A novel genetic system to detect protein-protein interactions. (1989) Nature, 340: 245-246.

7. Colas, P. and Brent, R. The impact of two-hybrid and related methods on biotechnology. (1998) Trends Biotechnol., 16; 355-363.

8. Broder, Y. C., Katz, S., and Aronheim, A. The Ras recruitment system, a novel approach to the study of protein-protein interactions. (1998) Curr. Biol. 8: 1121-1124.

9. Aronheim, A., Zandi, E., Hennemann, H., Elledge, S., and Karin, M. Isolation of an AP-1 repressor by a novel method for detecting protein-protein interactions. (1997) Mol. Cell. Biol., 17: 3094-3102.

10. Mumberg, D., Muller. R., and Funk, M. Regulatable promoters of Saccharomyces cerevisiae: comparison of transcriptional activity and their use for heterologous expression. (1994) Nuc. Acids Res., 22: 5767-5763.

11. Aronheim, A., Broder, Y. C., Cohen, A., Fritsch, A., Belisle, B., and Abo, A. Chp, a homologue of the GTPase Cdc42, activates the JNK pathway and is implicated in reorganizing the actin cytoskeleton. (1998) Curr. Biol., 8: 1125-1128.

12. Robzyk, K. and Kassir, Y. A simple and highly efficient procedure for rescuing autonomous plasmids from yeast. (1992) Nuc. Acids Res., 20: 3790.

13. Hancock, J. F., Magee. A. I., Childs, J., and Marshall, C. J. All ras proteins are polyisoprenylated but only some are palmitoylated. (1989) Cell, 57: 1167-1177.

14. Petitjean, A., Higler, F., and Tatchell, K. Comparison of thermosensitive alleles of the CDC25 gene involved in the cAMP metabolism of Saccharomyces cerevisiae. (1990) Genetics, 124: 797-806.

15. Aronheim, A. Improved efficiency Sos recruitment system; expression of the mammalian GAP reduces isolation of Ras GTase false positives. (1997) Nuc. Acids Res., 25: 3373-3374.

16. Marshall, C. Ras effectors. (1996) Curr. Opin. in Cell Biol., 8: 197-204.

17. Chevray, P. M. and Nathans. D. Protein interaction cloning in yeast: identification of mammalian proteins that react with the leucine zipper of Jun. (1992) Proc. Natl. Acad. Sci. USA., 89: 5789-5793.

18. Chinnaiyan, A. M., O'Rourke, K., Tewari, M., and Dixit, V. M. FADD, a novel death domain-containing protein, interacts with the death domain of Pas and initiates apoptosis. (1995) Cell, 81: 505-512.

19. Wallach, D., Boldin, M. P., Kovalenko, A. V., Malinin, N. L., Mett I. L., and Camonis, J. H. The yeast two-hybrid screening technique and its use in the study of protein-protein interactions in apoptosis. (1998) Curr Opin Immunol., 10: 131-136.

20. Furlanetto, R. W., Dey, B. R., Lopaczynski, W., and Nissley, S. P. 14-3-3 proteins interact with the insulin-like growth factor receptor but not the insulin receptor. (1997) Biochem J., 327: 765-71.

21. Dey, B. R., Furlanetto, R. W., and Nissley, S. P. Cloning of human p55 gamma, a regulatory subunit of phosphatidylinositol 3-kinase, by a yeast two-hybrid library screen with the insulin-like growth factor-I receptor. (1998) Gene, 209: 175-183.

22. Xu, P., Jacobs, A. R., and Taylor, S. I. Interaction of insulin receptor substrate 3 with insulin receptor, insulin receptor-related receptor, insulin-like growth factor-I receptor, and downstream signaling proteins. (1999) J. Biol Chem., 274: 15262-15270.

23. Van Aeist, L., Barr, M., Marcus, S., Polverino A., and Wigler, M. Complex formation between Ras and Raf and other protein kinases. (1993) Proc. natl. Acad. Sci. USA., 90: 6213-6217.

24. Juberty, G., Perlungher, R. R., and Macara, I. G. The Borgs, a new family of Cdc42 and TC10 GTPase-interacting proteins. (1999) Mol. Cell. Biol., 19: 6585-6597.

25. Johnsson, N. and Varshavski, A. Split ubiquitin as a sensor of protein interactions in vivo, (1994) Proc. Natl. Acad. Sci. USA., 91: 10340-10344.

26. Ozawa, T., Nogami, S., Sato, M., Ohya, Y., and Umezawa, Y. A fluorescent indicator for detecting protein-protein interactions in vivo based on protein splicing. (2000) Anal. Chem. 72: 5151-5157.

27. Ehrhard, K. N., Jacoby, J. J,, Fu, X.-Y., Jahn, R., and Dohlman, H. G. Use of G-protein fusions to monitor integral membrane protein-protein interactions in yeast. (2000) Nature Biotec. 2000, 18: 1075-1079.

28. Rocheville, M., Lange, D. C., Kumar, U., Patel, S. C., Patel, R. C., and Patel, Y. C. Receptors for dopamine and somatostatin: formation of hetero-oligomers with enhanced functional activity. (2000) Science, 288: 154-157.

What is claimed is:

1. A method of identifying interactions between polypeptides comprising:
(a) expressing in a cell substantially lacking Ras activity:
(i) a first polynucleotide encoding a first polypeptide being capable of interacting with a plasmalemma of the cell; and
(ii) a second polynucleotide encoding a second polypeptide fused to a cytoplasmic Ras mutant, said cytoplasmic Ras mutant being capable of said Ras activity if mobilized to said plasmalemma of said cell; and
(b) detecting a presence or absence of said Ras activity in said cell, said Ras activity being indicative of a possible interaction between said first polypeptide and said second polypeptide.

2. The method of claim 1, wherein said first polypeptide is a native membrane protein.

3. The method of claim 1, further comprising:
(c) independently expressing in said cell substantially only said second polynucleotide to thereby determine if said Ras activity detected in step (b) is dependent on said interaction between said first polypeptide and said second polypeptide.

4. The method of claim 3, wherein said first polypeptide is a native membrane protein.

5. The method of claim 3, wherein an expression of said first polynucleotide and/or said second polynucleotide in said cell is separately regulatable such that step (c) is effected by downregulating an expression of said first polynucleotide and/or upregulating an expression of said second polynucleotide.

6. The method of claim 1, wherein said cell substantially lacking Ras activity is a yeast cell exhibiting a mutant Ras phenotype characterized by growth suppression under non-permissive conditions.

7. The method of claim 6, wherein said cytoplasmic Ras mutant is capable of complementing said mutant Ras phenotype if mobilized to said plasmalemma of said cell.

8. The method of claim 1, wherein said first polypeptide includes an amino acid sequence for plasmalemma targeting.

9. A method of identifying interactions between polypeptides comprising:
(a) expressing in cells substantially lacking Ras activity:
(i) a first polynucleotide encoding a first polypeptide being capable of interacting with a plasmalemma of said cells; and
(ii) a library of polynucleotides each encoding a distinct polypeptide used to fused to a cytoplasmic Ras mutant, said cytoplasmic Ras mutant being capable of said Ras activity if mobilized to said plasmalemma of said cells; and
(b) identifying a subset of cells exhibiting said Ras activity, said Ras activity being indicative of a possible interaction between said first polypeptide and a distinct polypeptide expressed in said subset of cells.

10. The method of claim 9, further comprising isolating from each cell of said subset of cells a polynucleotide encoding said distinct polypeptide.

11. The method of claim 9, wherein said first polypeptide is a native membrane protein.

12. The method of claim 9, further comprising:
(c) independently expressing in said cells substantially only said library of polynucleotides to thereby determine if said Ras activity exhibited in said subset of cells is dependent on an interaction between said first polypeptide and said distinct polypeptide.

13. The method of claim 12, wherein said first polypeptide is a native membrane protein.

14. The method of claim 12, wherein an expression of said first polynucleotide and/or said library of polynucleotides in said cells is separately regulatable such that step (c) is effected by downregulating an expression of said first polynucleotide and/or upregulating an expression of said library of polynucleotides.

15. The method of claim 9, wherein said cells substantially lacking Ras activity are yeast cells exhibiting a mutant Ras phenotype characterized by growth suppression under non-permissive conditions.

16. The method of claim 15, wherein said cytoplasmic Ras mutant is capable of complementing said mutant Ras phenotype if mobilized to said plasmalemma of said cells.

17. The method of claim 9, wherein said first polypeptide includes an amino acid sequence for plasmalemma targeting.

18. A method of identifying interactions between polypeptides comprising:
(a) expressing in cells substantially lacking Ras activity:
(i) a library of polynucleotides each encoding a first polypeptide being capable of interacting with a plasmalemma of said cells fused to a second polypeptide; and
(ii) a second polynucleotide encoding a cytoplasmic Ras mutant fused to a third polypeptide, said cytoplasmic Ras mutant being capable of said Ras activity if mobilized to said plasmalemma of said cells; and
(b) identifying a subset of cells exhibiting said Ras activity, said Ras activity being indicative of a possible interaction between said third polypeptide and said second polypeptide expressed in each cell of said subset of cells.

19. The method of claim 18, further comprising isolating from each cell of said subset of cells a polynucleotide encoding said second polypeptide.

20. The method of claim 18, wherein said first polypeptide is a native membrane protein.

21. The method of claim 18, further comprising:
(c) independently expressing in said cells substantially only said second polynucleotide to thereby determine if said Ras activity exhibited in said subset of cells is dependent on an interaction between said third polypeptide and said second polypeptide.

22. The method of claim 21 wherein said first polypeptide is a native membrane protein.

23. The method of claim 21, wherein an expression of said library of polynucleotides and/or said second polynucleotide in said cells is separately regulatable such that step (c) is effected by downregulating an expression of said library of polynucleotides and/or upregulating an expression of said second polynucleotide.

24. The method of claim 18, wherein said cells substantially lacking Ras activity are yeast cells exhibiting a mutant Ras phenotype characterized by growth suppression under non-permissive conditions.

25. The method of claim 24, wherein said cytoplasmic Ras mutant is capable of complementing said mutant Ras phenotype if mobilized to said plasmalemma of said cells.

26. The method of claim 18, wherein said first polypeptide includes an amino acid sequence for plasmalemma targeting.

27. A method of identifying interactions between polypeptides comprising:
(a) expressing in cells substantially lacking Ras activity:
(i) a first library of polynucleotides each encoding a first polypeptide being capable of interacting with a plasmalemma of said cells fused to a second polypeptide; and
(ii) a second library of polynucleotides each encoding a cytoplasmic Ras mutant fused to a third polypeptide, said cytoplasmic Ras mutant being capable of said Ras activity if mobilized to said plasmalemma of said cells; and
(b) identifying a subset of cells exhibiting said Ras activity, said Ras activity being indicative of a possible interaction between said third polypeptide and said second polypeptide expressed in each cell of said subset of cells.

28. The method of claim 27, further comprising isolating from each cell of said subset of cells polynucleotides encoding said second polypeptide and said third polynucleotides.

29. The method of claim 27, wherein said first polypeptide is a native membrane protein.

30. The method of claim 27, further comprising:
(c) independently expressing in said cells substantially only said second library of polynucleotides to thereby determine if said Ras activity exhibited in said subset of cells is dependent on an interaction between said third polypeptide and said second polypeptide.

31. The method of claim 30, wherein said first polypeptide is a native membrane protein.

32. The method of claim 30, wherein an expression of said first library of polynucleotides and/or said second library of polynucleotides in said cells is separately regulatable such that step (c) is effected by downregulating an expression of said first library of polynucleotides and/or upregulating an expression of said second library of polynucleotides.

33. The method of claim 27, wherein said cells substantially lacking Ras activity are yeast cells exhibiting a mutant Ras phenotype characterized by growth suppression under non-permissive conditions.

34. The method of claim 33, wherein said cytoplasmic Ras mutant is capable of complementing said mutant as phenotype if mobilized to said plasmalemma of said cells.

35. The method of claim 27, wherein said first polypeptide includes an amino acid sequence for plasmalemma targeting.

36. A nucleic acid construct comprising a polynucleotide encoding a cytoplasmic Ras mutant under the transcriptional control of an inducible promoter.

37. The nucleic acid construct of claim 36, wherein said inducible promoter is functional in eukaryotic cells.

38. The nucleic acid construct of claim 36, wherein said inducible promoter is selected from the group consisting of a Gal1 promoter, a Yes2 promoter, a Met425 promoter and a copper inducible promoter.

39. A nucleic acid construct library comprising a plurality of nucleic acid constructs each including a first polynucleotide region encoding a cytoplasmic Ras mutant translationally fused to a second polynucleotide region encoding a distinct polypeptide.

40. The nucleic acid construct library of claim 39, wherein each of said plurality of nucleic acid constructs further includes a promoter for directing the expression of said first and said second polynucleotide regions.

41. The nucleic acid construct library of claim 40, wherein said promoter is functional in eukaryotic cells.

42. The nucleic acid construct library of claim 40, wherein said promoter is an inducible promoter selected from the group consisting of a Gal1 promoter, a Yes2 promoter, a Met425 promoter and a copper inducible promoter.

43. A nucleic acid construct system comprising:
(a) a first nucleic acid construct including a first polynucleotide encoding a first polypeptide being capable of interacting with a plasmalemma of a cell in which it is expressed; and
(b) a second nucleic acid construct including a second polynucleotide encoding a cytoplasmic Ras mutant fused to a second polypeptide.

44. The nucleic acid construct system of claim 43, wherein said first and said second nucleic acid construct each further include a promoter sequence for directing an expression of said first and said second polynucleotides in a eukaryotic cell.

45. The nucleic acid construct system of claim 44, wherein said first nucleic acid construct includes a first promoter sequence and said second nucleic acid construct includes a second promoter sequence, whereas said first and said second promoter sequences are independently selected from the group consisting of a constitutive promoter sequence and an inducible promoter sequence.

46. The nucleic acid construct system of claim 45, wherein said inducible promoter sequence is selected from the group consisting of a Gal1 promoter, a Yes2 promoter, a Met425 promoter and a copper inducible promoter.

47. A method of identifying polypeptides of interest comprising:
(a) expressing in a cell substantially lacking Ras activity:
(i) a first polynucleotide encoding a first polypeptide being capable of modifying a plasmalemma of the cell; and
(ii) a second polynucleotide encoding a second polypeptide fused to a cytoplasmic Ras mutant, said cytoplasmic Ras mutant being capable of said Ras activity if mobilized to said plasmalemma of said cell; and
(b) detecting a presence or absence of said Ras activity in said cell, said Ras activity being indicative of membrane mobilization of said second polypeptide resultant from a modification to said plasmalemma effected by said first polypeptide.

48. The method of claim 47, wherein said modifying said plasmalemma of the cell is effected by modifying a protein or phospholipid component of said plasmalemma.

49. The method of claim 47, wherein said first polypeptide is PI3kinase.


SEQUENCE LISTING (7 sequences)

SEQ ID NO: 1 - DNA, 561 bp, Rattus rattus
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggcctggaggagtacagcgccatgcgggaccagtacatgcgcaccggggagggcttcctgtgtggtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagggaattc

SEQ ID NO: 2 - DNA, 711 bp, Rattus rattus
atgccgccgcgggagctgagcgaggccgagccaccgcctctcccggcctcgacccctcctccgcggcggcgcagcgcccctccggagctgggcatcaaatgcgtgctggtgggcgacgtcgcggtgggcaagagcagcctcatcgtcagctacacctgcaatggatacccctcgcgctatcggcctacagcactggacactttctccgtgcaagtcctggtagatggagcccctgtgcgaattgagctctgggacacagcagggcaggaggactttgaccggcttcgttctctctgctacccggataccgatgtctttctggcttgcttcagcgtggtgcagcccagctcctttcaaaacataacagaaaaatggctgccggagatccgcactcacaacccccaagcacctgtgttgctggtgggcactcaggccgacctgagggacgatgtcaatgtactaattcagttggaccaaggaggtcgggagggcccagtacccgaaccccaagcccagggtttggctgagaagatccgggcctgctgctaccttgagtgctcagccttgacgcagaagaacttgaaggaggtgttcgactcggccattctcagtgcgattgagcacaaagcccgcctggagaagaaactgaacgcaaaaggtgtgcgcacgctctctcgctgtcgctggaagaagttcttctgctttgtttga

SEQ ID NO: 3 - DNA, 24 bp, Artificial
  synthetic oligonucleotide
cggaattcaaatgcgtgctggtgg

SEQ ID NO: 4 - DNA, 20 bp, Artificial
  synthetic oligonucleotide
ccaagctatttaggtgacac

SEQ ID NO: 5 - DNA, 33 bp, Unknown
  myc epitope tag
atggtgcagaagctgatctccgaggaggacctg

SEQ ID NO: 6 - DNA, 86 bp, Unknown
  v-Src myristoylation sequence
atggggagtagcaagagcaagcctaaggaccccagccagcgccggcccggagatccactagtaacggccgccagtgtgctggaatt

SEQ ID NO: 7 - DNA, 63 bp, Unknown
  CAAX box consensus sequence
aagctgaaccctcctgatgagagtggccccggctgcatgagctgcaagtgtgtgctctcctga